(19) United States
(12) Patent Application Publication
(10) Pub. No.: US 2019/0030355 A1
(43) Pub. Date: Jan. 31, 2019

(54) Method for Increasing and Sustaining Free Radical Levels in Tissue of Interest

(71) Applicant: ROBERT E. SANDSTROM, Longview, WA (US)

(72) Inventor: ROBERT E. SANDSTROM, Longview, WA (US)

(21) Appl. No.: 16/072,711

(22) PCT Filed: Jan. 25, 2017
(86) PCT No.: PCT/US17/14923

§ 371 Date: Jul. 25, 2018

Related U.S. Application Data
(60) Provisional application No. 62/286,474, filed on Jan. 25, 2016.

(51) Int. Cl. A61N 2/00; A61K 33/24; A61K 41/00; A61K 51/12; A61N 5/10

(52) U.S. Cl. CPC A61N 2/002; A61K 33/24; A61K 39/00; A61K 51/1244; A61N 5/1027; A61K 41/0057

(57) ABSTRACT

A method of creating and sustaining an elevated level of free radicals in a volume of targeted tissue that utilizes targeted nanostructures (16) that include a metallic component (26) that acts to amplify the effects of a free radical-producing stimulus; a magnetic component; and a binding component (24) that acts to bind to cellular components present in the targeted tissue. To practice the method, the targeted nanostructures are introduced into the targeted tissue and a free radical-producing stimulus, which may be in the form of a particle beam (20) is provided at the targeted tissue volume.

### FIELD OF THE INVENTION

The present invention is in the field of methods of increasing and sustaining free radical levels in a targeted tissue of interest. More specifically, the field of the invention is in the field of methods utilizing metal loaded nanoparticles and a free radical-producing stimulus.

### BACKGROUND ART

A magnetic field of appropriate field strength affects the reaction dynamics of free radicals by quantum mechanisms which alter the spin dynamics of the free radicals. The radicals precess by a mechanism comparable to Larmor Precession. Free radicals are produced when tissues are irradiated by external photon, electron or particle beams or by exposure to a drug or therapeutic agent. Some chemotherapeutic agents employed in tumor treatment also produce their effect largely on the basis of free radical mechanisms. A magnetic field alters the reaction dynamics and results in increased cell injury when tissues and specifically tumor tissues are exposed to a structured magnetic field designed to treat the tissue exposed to an induced free radical concentration.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first separate aspect, the present invention may take the form of a method of creating and sustaining an elevated level of free radicals in a volume of targeted tissue that utilizes targeted nanostructures that include a metallic component that acts to amplify the effects of a free radical-producing stimulus; a magnetic component; and a binding component that acts to bind to cellular components present in the targeted tissue. To practice the method, the targeted nanostructures are introduced into the targeted tissue and a free radical-producing stimulus is provided at the targeted tissue volume.

In a second separate aspect, the present invention may take the form of a nanostructure adapted to facilitate treatment of targeted tissue, comprising a metallic component that acts to amplify the effects of free radical-producing stimulus; a magnetic component; and a binding component that binds to cellular features present in the targeted tissue.

In a third separate aspect, the present invention may take the form of a method of creating and sustaining an elevated level of free radicals in a volume of targeted tissue that uses targeted nanostructures that include a metallic component that acts to amplify the effects of a free radical-producing stimulus, and a binding component that acts to bind to cellular components present in the targeted tissue. To practice the method, the targeted nanostructures are introduced into the targeted tissue and a free radical-producing stimulus is performed at the targeted tissue. Further, a magnetic field is created and shaped to cover the targeted tissue and has a field strength of between 1 and 300 gauss at the targeted tissue, thereby extending lifetimes of free radicals in the targeted tissue.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1 is an illustration of a pair of lungs, showing a tumor by a closed form.

FIG. 2 is the same view as FIG. 1, with the tumor treated with Nanoparticles. An inset shows a very greatly expanded view of the tissues, with the nanoparticles binding to sites on the nuclear membranes of the cells.

FIG. 3 shows the tumor being irradiated with an external radiation beam.

FIG. 4 is an illustration of the effects of irradiation and a low level magnetic field, inside a cell or at the cell surface.

### PREFERRED MODES OF CARRYING OUT THE INVENTION

### Definition

In the context of this application, a “particle beam” is any beam of particles, including electrons or photons, in addition to charged particles.

In one preferred embodiment, a combination of irradiation and a low intensity magnetic field exposure produces enhances cell death through the use of metallic nanostructures. This term encompasses quantum wells, nanotubes and nanowires, and quantum dots, which can be formulated to incorporate metals, including gold, copper, silver, titanium, zinc or cadmium or technetium. In one preferred embodiment tissue is treated with zinc loaded, tissue targeted nanostructures that enhance free radical dynamics in irradiated tissue. In an additional preferred embodiment, chemotherapeutic agents, including but not limited to bleomycin, adriamycin and platinum compounds which act through free radical mechanisms induce an enhanced cell death and apoptosis when the tumor is treated with localized metallic nanomaterials which increase the effective cellular free radical concentration. Nanomaterials and specifically those incorporating metallic nanostructures enhance the free radical effect by increasing the effective concentration of free radicals in the tumor. The nanomaterials can be localized to the tumor by methods that include antibody tumor cell specific binding. The result is increased cell death in tissues jointly treated with the magnetic field, induced free radicals and tumor localized and concentrated nanometallic materials.

When nanoparticles are used, in preferred embodiments they include antibodies or aptamers that selectively attach to specific cell components, such as the cell wall, nucleoplasm, mitochondria, or ribosomes, or some combination of cell components. In a preferred method, a liquid suspension of nanoparticles is injected at a venous site, or alternatively, directly into the target tissue. Selective attachment can be accomplished by attaching a metallic portion of the nanoparticles to a tumor specific antibody or aptamer e.g. carcinoembryonic antigen (CEA), prostate-specific antigen (PSA) or synthetic antibody-like congeners. In a preferred embodiment, selective concentration of the nanoparticles is effected by injection, painting, or by using localized magnetic fields to concentrate magnetic particles. In an additional preferred embodiment, an endoscopic method or a trans vascular method is used to introduce nanomaterials at a specific anatomic site, which may be a tumor site.

In some preferred embodiments, anatomic localization of the nanomaterials is confirmed by imaging, for example: ultrasound imaging, computerized axial tomography, magnetic resonance imaging or specifically designed nuclear medicine imaging employing labelled nanomaterials. In one preferred method nanomaterials are concentrated at an anatomic location, which is then imaged. The resultant image information is then used to conform a magnetic field of selective strength to that anatomic location. Contemporaneously, the anatomic location is treated with external beam or by implanting a radiation source for therapeutic purposes in or near the anatomic site of the tumor or by targeting an anatomic site of interest with a radioisotope loaded nanostructure that induces free radicals. In a preferred embodiment, the radioisotope is iodine-131.

Metals that may be used in the nanostructures include high Z metals, such as copper, gold and zinc or transition metals from groups 3 through 12 of the periodic table of elements.

The advantages of the methods described above include enhanced free radical reactivity in tissue treated with a magnetic field conformed to the irradiated area at a selective magnetic field strength. Additional benefit is derived from nanomaterials that localize and increase free radical activity in treated tissue e.g. a treated tumor. The use of imaging to define the anatomic location, to confirm the localization of the employed nanomaterials, to design and confirm the magnetic field and define and confirm a selective magnetic field strength and guide the irradiation of the anatomic site provides further benefit.

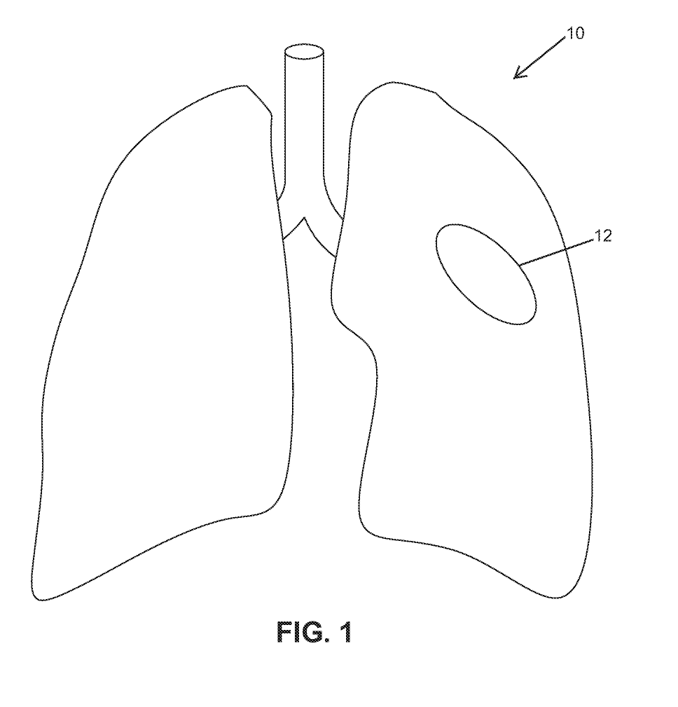

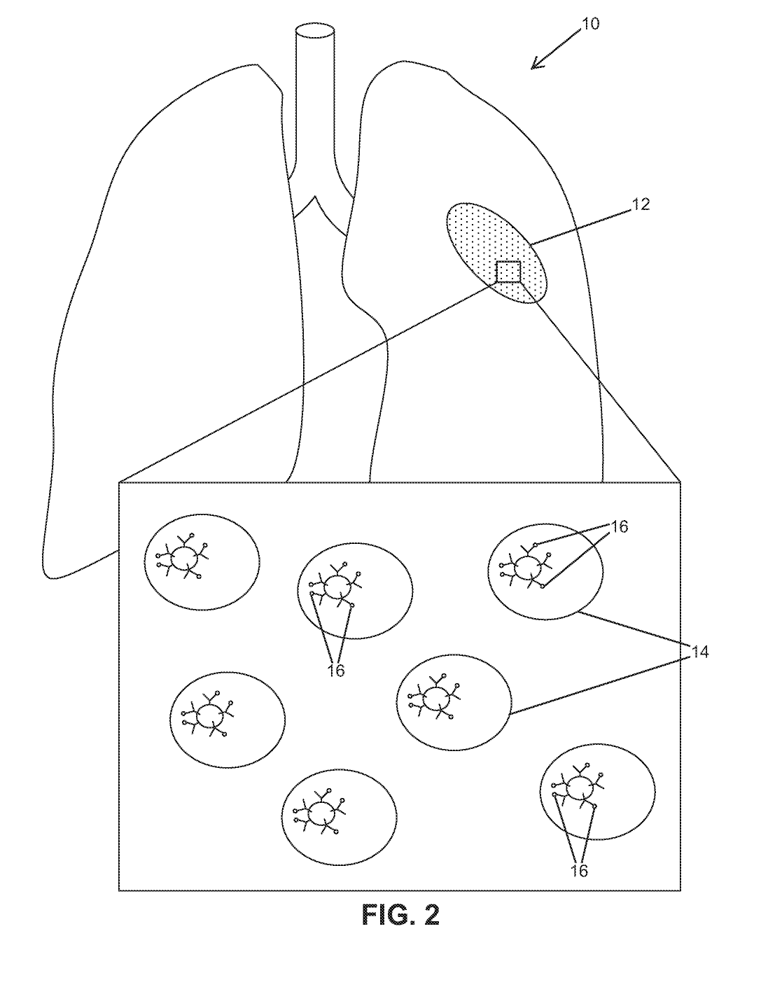

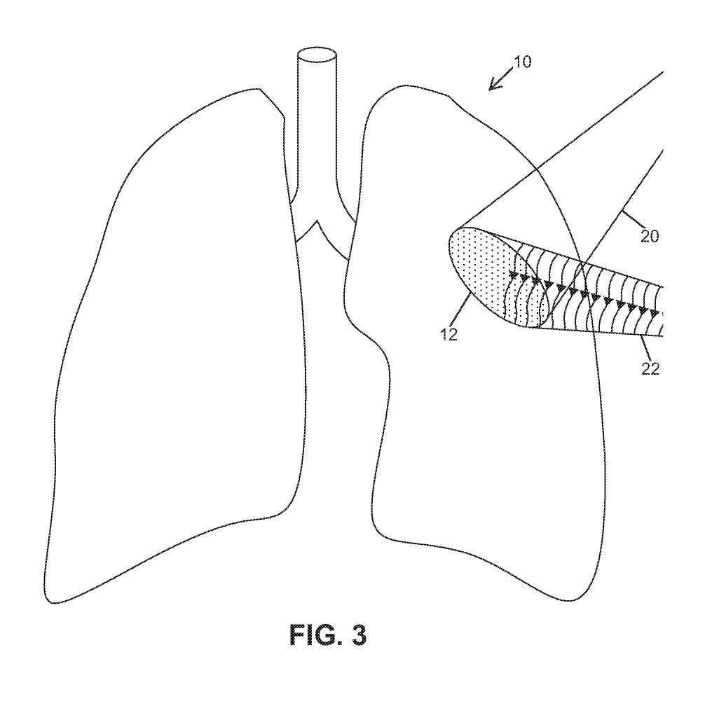

Referring to FIGS. 1-3, in one preferred method, a patient 10 with a cancer known to be carcinoembryonic antigen positive as proven by prior biopsy and analysis is infused with a liquid suspension of platinum nanostructures 16 which incorporates a carcinoembryonic binding site which selectively binds to the nuclear membrane of the cells 14 of the tumor. The tumor is visualized with standard imaging modalities e.g. computerized axial tomography. On the basis of the imaged tumor a radiation treatment plan is developed to employ conformal radiation therapy including intensity modulated radiation therapy external beam 20 cobalt irradiation at a total dose of 102 Gy. Prior to irradiation a magnetic field 22 is structured at a specific uniform field strength, usually of 38 gauss. In an alternative preferred embodiment, nanostructures 16 include magnetic material, such as ferromagnetic material, that collectively produces a magnetic field 22.

The magnetic field 22 conforms to the tumor anatomy to be irradiated and is monitored to establish the field strength and anatomic conformity. The patient then undergoes planned irradiation. The presence of the magnetic field 22 coupled with the metallic platinum nanostructures 16 results in enhanced tumor cell death in the irradiated tumor. The localization of the platinum nanostructures 16 to tumor cells and the anatomic conformity of the magnetic field concentrate the free radical damage to the tumor and mitigates injury to normal adjacent tissue.

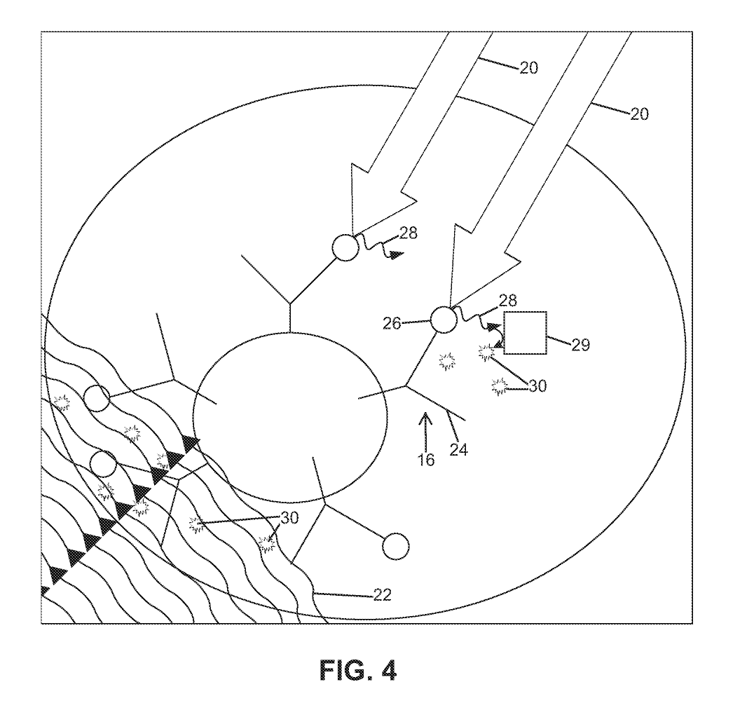

Referring to FIG. 4, and in more general terms when the radiation 20 strikes the metal portion 26 of nanostructures 16 (held in place by binding portion 24), emissions 28 are created. In one preferred embodiment emissions 28 are photons. Emissions 28 strike molecules 29, creating free radicals 30. Free radicals 30 are sustained by magnetic field 22. For clarity of presentation magnetic field 22, as well as radiation 20, are shown as being localized, although each would cover the cell 14. The metal nanostructure 16 acts as a radiation multiplier locally by increasing free radical concentration and reactivity.

### INDUSTRIAL APPLICABILITY

The present invention finds industrial applicability in the design and production of nanostructures.

The present invention may include the following forms:

A method of creating and sustaining an elevated level of free radicals in a volume of targeted tissue that utilizes targeted nanostructures that include a metallic component that acts to amplify the effects of a free radical-producing stimulus; a magnetic component; and a binding component that acts to bind to cellular components present in the targeted tissue. To practice the method, the targeted nanostructures are introduced into the targeted tissue and a free radical-producing stimulus is provided at the targeted tissue volume.

Preferred methods may include the following features, either singly or in combination: The metal portion includes zinc. The free radical-producing stimulus is an externally-created particle beam directed at said targeted tissue; The particle beam is comprised of electrons; The particle beam is comprised of photons; The particle beam is comprised of charged particles; The magnetic components of the nanostructures collectively produce a magnetic field that has a strength of between 1 and 300 Gauss; The binding portion is an antibody; The binding portion is an aptamer; The target tissue is cancerous; The metal portion includes a metal selected from a group consisting essentially of zinc, cadmium and technetium; The metal portion includes a metal selected from a group consisting essentially of copper, silver and gold; The cellular features are on the nuclear membranes of cells of said target tissue; The cellular features are on the cell membranes of cells of said target tissues; The nanostructures further include a radioisotope that serves as the free radical-producing stimulus; The radioisotope is iodine-131; The magnetic component comprises ferromagnetic material; Introducing the nanoparticles into the targeted tissue, includes providing a liquid suspension of said nanoparticles and injecting said nanoparticles into a venous site; and/or Introducing said nanoparticles into said targeted tissue, includes providing a liquid suspension of said nanoparticles and injecting said liquid suspension on nanoparticles directly into said targeted tissue.

A nanostructure adapted to facilitate treatment of targeted tissue, comprising a metallic component that acts to amplify the effects of free radical-producing stimulus; a magnetic component; and a binding component that binds to cellular features present in the targeted tissue.

Preferred nanostructures may include the following features, either singly or in combination: The metal portion includes zinc. The magnetic components of the nanostructures collectively produce a magnetic field that has a strength of between 1 and 300 Gauss; The binding portion is an antibody; The binding portion is an aptamer; The target tissue is cancerous; The metal portion includes a metal selected from a group consisting essentially of zinc, cadmium and technetium; The metal portion includes a metal selected from a group consisting essentially of copper, silver and gold; The cellular features are on the nuclear membranes of cells of said target tissue; The cellular features are on the cell membranes of cells of said target tissues; The nanostructures further include a radioisotope that serves as the free radical-producing stimulus; and/or The radioisotope is iodine-131; The magnetic component comprises ferromagnetic material.

A method of creating and sustaining an elevated level of free radicals in a volume of targeted tissue that uses targeted nanostructures that include a metallic component that acts to amplify the effects of a free radical-producing stimulus, and a binding component that acts to bind to cellular components present in the targeted tissue. To practice the method, the targeted nanostructures are introduced into the targeted tissue and a free radical-producing stimulus is performed at the targeted tissue. Further, a magnetic field is created and shaped to cover the targeted tissue and has a field strength of between 1 and 300 gauss at the targeted tissue, thereby extending lifetimes of free radicals in the targeted tissue.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method of creating and sustaining an elevated level of free radicals in a volume of targeted tissue comprising:
(a) providing targeted nanostructures that include:
(i) a metallic component that acts to amplify the effects of a free radical-producing stimulus;
(ii) a magnetic component; and
(iii) a binding component that acts to bind to cellular components present in said targeted tissue;
(b) Introducing said targeted nanostructures into said targeted tissue; and
(c) providing a free radical-producing stimulus at said targeted tissue.

2. The method of claim 1, wherein said metal portion includes zinc.

3. The method of claim 1, wherein said free radical-producing stimulus is an externally-created particle beam directed at said targeted tissue.

4. The method of claim 3, wherein said particle beam is comprised of electrons.

5. The method of claim 3, wherein said particle beam is comprised of photons.

6. The method of claim 3, wherein said particle beam is comprised of charged particles.

7. The method of claim 1, wherein said magnetic components of said nanostructures collectively produce a magnetic field that has a strength of between 1 and 300 Gauss.

8. The method of claim 1, wherein said binding portion is an antibody.

9. The method of claim 1, wherein said binding portion is an aptamer.

10. The method of claim 1, wherein said target tissue is cancerous.

11. The method of claim 1, wherein said metal portion includes a metal selected from a group consisting essentially of zinc, cadmium and technetium.

12. The method of claim 1, wherein said metal portion includes a metal selected from a group consisting essentially of copper, silver and gold.

13. The method of claim 1, wherein said cellular features are on the nuclear membranes of cells of said target tissue.

14. The method of claim 1, wherein said cellular features are on the cell membranes of cells of said target tissues.

15. The method of claim 1, wherein said nanostructures further include a radioisotope that serves as the free radical-producing stimulus.

16. The method of claim 15, wherein said radioisotope is iodine-131.

17. The method of claim 1, wherein said magnetic component comprises ferromagnetic material.

18. The method of claim 1, wherein introducing said nanoparticles into said targeted tissue, includes providing a liquid suspension of said nanoparticles and injecting said nanoparticles into a venous site.

19. The method of claims 1, wherein introducing said nanoparticles into said targeted tissue, includes providing a liquid suspension of said nanoparticles and injecting said liquid suspension on nanoparticles directly into said targeted tissue.

20. A nanostructure adapted to facilitate treatment of targeted tissue, comprising:
(a) a metallic component that acts to amplify the effects of free radical-producing stimulus;
(b) a magnetic component; and
(c) a binding component that binds to cellular features present in said targeted tissue.

21. The nanostructure of claim 20, wherein said metal portion includes zinc.

22. The nanostructure of claim 20, wherein said binding portion is an antibody.

23. The nanostructure of claim 20, wherein said binding portion is an aptamer.

24. The nanostructure of claim 20, wherein said target tissue is cancerous.

25. The nanostructure of claim 20, wherein said metal portion includes a metal selected from a group consisting essentially of zinc, cadmium and technetium.

26. The nanostructure of claim 20, wherein said metal portion includes a metal selected from a group consisting essentially of copper, silver and gold.

27. The nanostructure of claim 20, wherein said cellular features are on the nuclear membranes of cells of said target tissue.

28. The nanostructure of claim 20, wherein said cellular features are on the cell membranes of cells of said target tissues.

29. The nanostructure of claim 20, further including a radioisotope that serves as a free radical-producing stimulus.

30. The nanostructure of claim 29, wherein said radioisotope is iodine-131.

31. The nanostructure of claim 20, wherein said magnetic component comprises ferromagnetic material.

32. A method of creating and sustaining an elevated level of free radicals in a volume of targeted tissue comprising:
(a) providing targeted nanostructures that include:
(i) a metallic component that acts to amplify the effects of a free radical-producing stimulus; and
(ii) a binding component that acts to bind to cellular components present in said targeted tissue;
(b) Introducing said targeted nanostructures into said targeted tissue;
(c) providing a free radical-producing stimulus at said targeted tissue; and
(d) creating a magnetic field shaped to cover said targeted tissue and having a field strength of between 1 and 300 gauss at said targeted tissue, thereby extending lifetimes of free radicals in said targeted tissue.
